(19) United States
(12) Patent Application Publication
(10) Pub. No.: US 2004/0193016 A1
(43) Pub. Date: Sep. 30, 2004

(54) Endoscopic delivery system for the non-destructive testing and evaluation of remote flaws

(76) Inventors: Thomas Root, Beverly, MA (US); Ralph Tillinghast, Hardwick, NJ (US); Matthew Maher, Worcester, MA (US); William LaFlash, Northbridge, MA (US); Robert Krupa, Leominster, MA (US)

Correspondence Address: MIRICK O'CONNELL, 1700 WEST PARK DRIVE, WESTBOROUGH, MA, 01581-3941, US

(21) Appl. No.: 10/746,331

(22) Filed: Dec. 23, 2003

Related U.S. Application Data
(63) Continuation-in-part of application No. 10/462,951, filed on Jun. 17, 2003.
(60) Provisional application No. 60/389,168, filed on Jun. 17, 2002.
(60) Provisional application No. 60/436,553, filed on Dec. 26, 2002.

(51) Int. Cl. A61B 1/005

(52) U.S. Cl. 600/146

(57) ABSTRACT

An endoscope for remotely viewing and testing relatively inaccessible regions of structures that are under stress when used, the endoscope having a mechanically-articulated articulating distal end. The endoscope includes an elongated shaft having a proximal end and a distal working end. There is a working channel located within the shaft and open at both ends. A remote material stress-testing probe is located at least partially within the working channel, and is adapted to contact the region to be tested. There is at least one light guide in the shaft for carrying light introduced into the proximal end to a remote viewing area proximate the distal end. The endoscope further includes a user-operable shaft tip steering mechanism for articulating the distal end of the shaft. The tip steering mechanism includes at least two rotatable drums, at least a pair of wires coupled to the drums, and also coupled to the tool's articulating distal end, for translating drum rotation into distal end articulation, a mechanical joystick moveable translationally and through 360 degrees rotationally, and a mechanism coupling the joystick to the drums, that mechanically translates motion of the joystick into rotation of the drums, wherein motion of the joystick in one plane causes rotation of only a first drum, and motion of the joystick in a perpendicular plane causes rotation of only a second drum, and movements of the joystick not wholly within these two planes causes rotation of both the first and second drums.

### CROSS REFERENCE TO RELATED APPLICATION

This application is a continuation in part of and claims priority of nonprovisional patent application Ser. No. 10/462,951, filed on Jun. 17, 2003 entitled “Mechanical Steering Mechanism for Borescopes, Endoscopes, Catheters, Guide Tubes, and Working Tools”, and its provisional patent application serial No. 60/389,168, filed Jun. 17, 2002, entitled “Mechanical Joystick Steering Mechanism for Borescopes, Endoscopes, Catheters, Guide Tubes, and Working Tools”; and also claims priority of provisional patent application serial No. 60/436,553, filed Dec. 26, 2002 entitled “Endoscopic Delivery System for Visual, Eddy Current, Ultrasonic, and Electrochemical Fatigue Sensors for the Nondestructive Testing and Evaluation of Remote Flaws”.

### FIELD OF THE INVENTION

The invention relates to industrial endoscopes for remote viewing and testing of devices.

### BACKGROUND OF THE INVENTION

Remote areas under stress must be inspected periodically in order to determine if the part or structure is in danger of failure, and if so, what course of action is necessary to prevent failure. Fatigue critical locations in remote areas such as turbine blades within an aircraft engine, weld joints in building structures, or the like, are visually inspected using endoscopic devices (such as an endoscope, borescope, fiberscope, or the like). The visual inspection of these critical locations, however, in many instances cannot definitively determine if an observed feature is indeed a crack in need of remedial action, or a tool mark, foreign object, or other mark on the part of interest that has little or no bearing on the part's ability to withstand future stress. Visual inspection, even with computer enhanced visual capabilities, remains a qualitative and subjective technique. Visual inspection scopes typically do not provide the use of other instruments for testing or remediation of a potential crack or other material problem caused by fatigue.

An articulating or bending section is found at the distal end of some endoscopes. This bending section is controlled at the proximal end by a mechanism. This mechanism allows the operator of the scope to direct the distal end into the desired areas in which the endoscope has been placed. Typically this mechanism is found in three versions: one-way, two-way or four-way articulation. This represents the directions that the distal end can be moved. A fourth variation, utilized only with a joystick mechanism, is all-way articulation. FIG. 1 demonstrates these configurations of the distal end.

The distal end is typically articulated by pulling on wires that are held inside the insertion tube portion of the endoscope. These wires are connected to swing arms or drums that are moved or rotated by knobs, wheels, triggers, or levers. FIG. 2 shows a typical endoscope 500 with four-way articulation. This endoscope consists of two knobs 280, 290 (or, alternately, two levers or wheels) that are turned individually or simultaneously to move distal end 310 into the desired position.

The movement of the direction of the distal tip of a remote imaging device, commonly referred to as articulation, is most often accomplished by pushing and/or pulling wires attached between the distal tip of the endoscope and a gear system in the proximal handle. Gears (e.g., capstans, rack and pinion, cams) within the handle are moved by the operator using levers or wheels connected to the gears. In four-way articulation, the endoscope deflection is in two independent, perpendicular planes (e.g., left-right and up-down). In order to view a particular area that requires travel in both planes of movement, the operator must actuate two levers or knobs, usually in succession. This is cumbersome and not an intuitive process. Alternatively, an electronic joystick is employed that converts the more intuitive joystick movement into an electrical signal that can be processed and converted into electrical signals that drive a motor (for one-way and two-way articulation in a single plane) or two motors (for four-way and all-way articulation). The drawback with this means of articulation is the endoscope handle is typically connected (via an umbilical or tether) to an external power supply and processing electronics for the joystick and motors. This limits the portability of the device and the operator's access to remote locations. Alternatively, the motors, electronics, and power supply (e.g., batteries) are contained within the handle, making the device heavy, large, and difficult and tiring to use. Additionally, the operator lacks the “tactile feel” or feedback inherent in a mechanically actuated device that is often necessary to sense the device's advancement or resistance.

### SUMMARY OF THE INVENTION

This invention features a visual fiberscope (endoscope, borescope, fiberscope are used synonymously in this disclosure, and sometimes called “scope”) that has an integral working channel which permits the use of miniature non-destructive testing probes and remediation tools in remote and normally inaccessible areas such as the internal areas of an engine, metal structures within the walls of a building, remote sections of a pipe, and the like.

By combining nondestructive testing (NDT) probes such as Eddy Current (EC) probes, Ultrasonic Transducer (UT) probes, or Electrochemical Fatigue Sensor (EFS) probes in conjunction with visual inspection, an objective assessment of the visual feature can be quantified and used to determine: 1) if the feature is indeed a flaw in the form of a crack or stress riser, 2) the depth and width of a crack, and 3) the thickness of material surrounding the crack. The use of NDT probes in conjunction with visual inspection can quantify the size of the flaw and verify that the flaw has been removed or effectively reduced in size so as not to cause failure of the part upon further stress.

In addition to electromagnetic (eddy current) and ultrasonic probes, the working channel in the scope permits the delivery of magnetic particles (for use in magnetic particle testing), the delivery of fluorescent dyes and inclusion of UV transmitting light guides (used in dye penetrant testing), the delivery of optoacoustic measurement probes and laser delivery optics, and the use of electrochemical probes, such as the electrochemical fatigue sensor disclosed in U.S. Pat. Nos. 5,419,201 and 6,026,691.

This invention, a visual borescope with a working channel, enables the operator to insert NDT probes down the shaft of the visual borescope for the further examination and evaluation of a suspected crack by techniques that permit reliable and quantifiable measurements of the size of the flaw and the thickness of material in the area of the suspected flaw. This eliminates the false positive results that plague visual examination; for example, the assessments of a tool mark as a crack. The NDT probes can also confirm the presence of a flaw where visual inspection is ambiguous, and can assess the efficacy of remedial action taken on a flaw (such as the wall thickness of a turbine blade that has been “blended”, a process by which the area in and around a crack is removed by grinding).

In one aspect, the invention includes a mechanism that moves the articulating end of the scope in all four directions within the nominal sphere of the distal end. This aspect uses a joystick lever approach to articulate the distal end tip. The mechanism is a two-axis, mechanically actuated device that allows the user to rotate two drums, cams, or gears (all termed herein “drums”). The particular type of drum used is based upon the diameter, length, and size of the tool. The drums are moved individually or simultaneously in either direction (e.g. clockwise or counter clockwise) by applying manual pressure to a joystick lever in the direction of desired articulation. This rotation pulls and/or pushes the wires connected to the distal end of the tool, causing the distal end to articulate to a desired position. This articulated movement permits the user to direct the view and/or placement of an instrument on the surface of an imaginary sphere. This invention relies upon the mechanical force generated at the joystick by the operator's hand, rather than relying on an electronic joystick that converts the joystick movement to an electrical signal, proportional to the joystick movement, that is used to drive an electronic motor or motors. This mechanical joystick, therefore, provides an intuitive direction with which the distal tip location can be interpolated based upon the joystick location. Additionally, the operator maintains a tactile sense or “feel” for the advancement through and the placement of the distal tip's environment.

This manual joystick mechanism is unique in that the joystick position is representative of the position of the distal tip of the tool, making operation of the tool much more intuitive and easier to use. In addition, this is the only mechanism that provides a nominally spherical surface of operation. This all-way articulation can be viewed as movement of the distal tip in an R-Theta (radius and angle) or spherical coordinate system. This is differentiated from typical four-way articulation, which is movement of the distal tip along two independent perpendicular planes (e.g., the XZ and YZ planes where the tool axis lies along the Z-axis). While both four-way and all-way articulation have similar end results (i.e., the distal tip can be moved to similar positions), only the all-way joystick mechanism accomplishes this in a simple, single step movement, whereas the four-way mechanism must make two independent movements to arrive at the same place in space.

This invention features an endoscope for remotely viewing and testing relatively inaccessible regions of structures that are under stress when used, the endoscope having a mechanically-articulated articulating distal end. The endoscope includes an elongated shaft having a proximal end and a distal working end. There is a working channel located within the shaft and open at both ends. A remote material stress testing probe is located at least partially within the working channel, and is adapted to contact the region to be tested. There is at least one light guide in the shaft for carrying light introduced into the proximal end to a remote viewing area proximate the distal end. The endoscope further includes a user-operable shaft tip steering mechanism for articulating the distal end of the shaft. The tip steering mechanism includes at least two rotatable drums, at least a pair of wires coupled to the drums, and also coupled to the tool's articulating distal end, for translating drum rotation into distal end articulation, a mechanical joystick moveable translationally and through 360 degrees rotationally, and a mechanism coupling the joystick to the drums, that mechanically translates motion of the joystick into rotation of the drums, wherein motion of the joystick in one plane causes rotation of only a first drum, and motion of the joystick in a perpendicular plane causes rotation of only a second drum, and movements of the joystick not wholly within these two planes causes rotation of both the first and second drums.

### BRIEF DESCRIPTION OF THE DRAWINGS

FIGS. 1A through 1D are schematic diagrams illustrating the four typical articulation modes of a tool with a distal articulating head of the type in which the invention is useful;

FIG. 2 is a schematic diagram of a prior art tool with an articulating distal end, showing one manner in which the user accomplishes articulation;

FIG. 3 is a partial schematic diagram of one preferred embodiment of the mechanism for articulating the distal end of an elongated tool of the invention;

FIG. 4 shows an alternative arrangement to the mechanism of FIG. 3;

FIG. 5 is yet another alternative arrangement for the articulation mechanism of the invention;

FIG. 6 is yet another alternative arrangement for the articulation mechanism of the invention;

FIG. 7 is still another alternative arrangement for the articulation mechanism of the invention;

FIGS. 8A and 8B are schematic views of one braking mechanism for the articulation mechanism of the invention;

FIG. 9 is a schematic view of another braking mechanism for the articulation mechanism of the invention;

FIG. 10 is a schematic view of yet another braking mechanism for the articulation mechanism of the invention;

FIG. 11 is a schematic view of yet another braking mechanism for the articulation mechanism of the invention;

FIG. 12 is a schematic, partially cutaway view of the preferred embodiment of the invention;

FIGS. 12A and 12B are enlarged views of detail A and detail B, respectively, of the preferred embodiment of FIG. 12;

FIG. 13 is a cross-sectional view of the elongated section of the preferred embodiment of FIG. 12;

FIG. 14 is an end view of the elongated section of the preferred embodiment of FIG. 12;

FIG. 15 is a partial schematic view of a non-destructive testing (NDT) probe for use with the embodiment shown in FIG. 12;

FIG. 16 is a partial schematic view of another NDT probe for use with the embodiment shown in FIG. 12;

FIG. 17 is a graph of crack detecting results using an eddy current NDT probe; and

FIG. 18 is a partial schematic cross-sectional view of another NDT probe for use with the embodiment shown in FIG. 12.

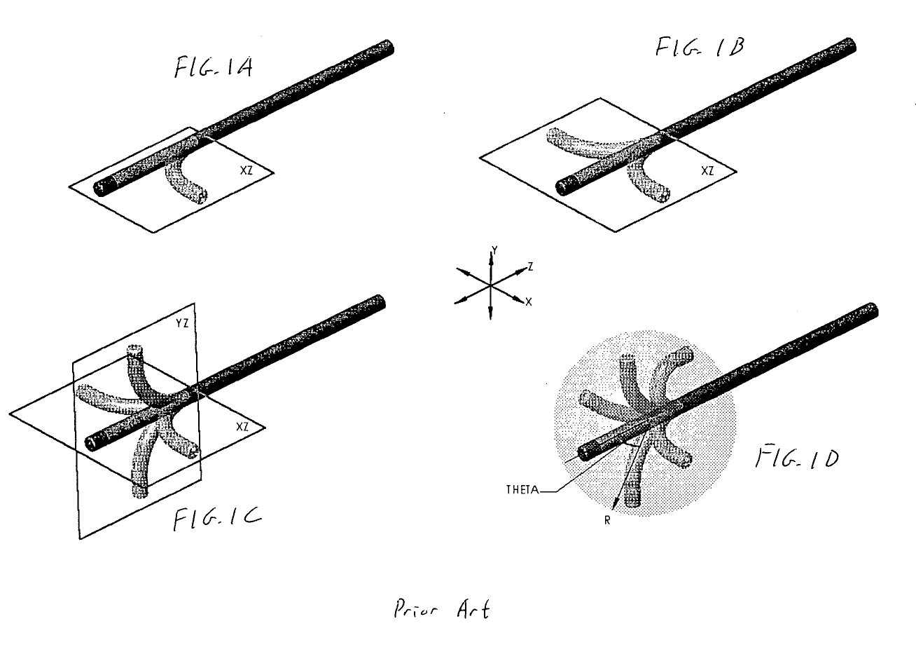

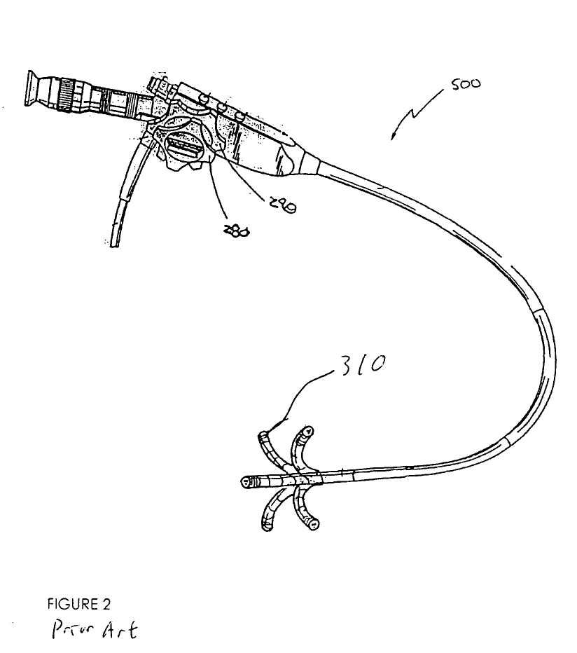

### DETAILED DESCRIPTION OF THE PREFERRED EMBODIMENTS OF THE INVENTION

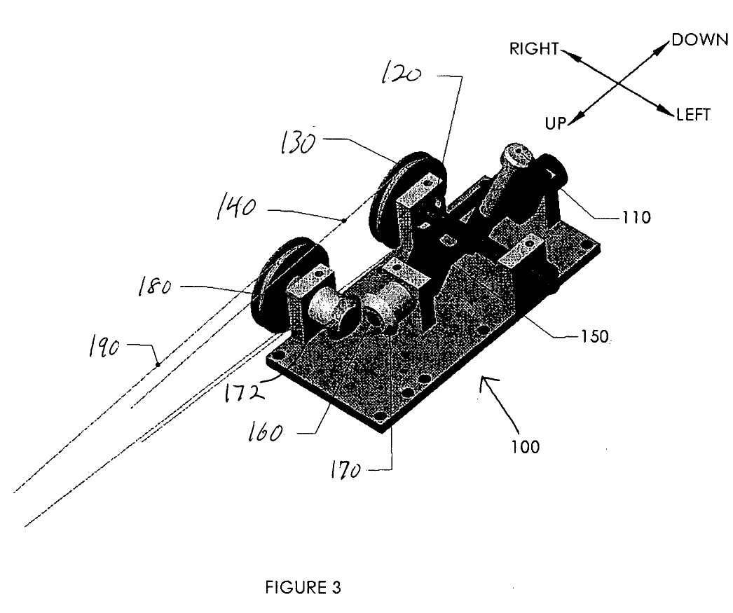

FIG. 3 shows a configuration of the preferred embodiment of the articulation mechanism for the invention. The following is a breakdown of each part of the articulation mechanism.

Articulation Section:

The articulation section of the device can employ several different means of controlling the direction of the articulation. One method employs vertebrae that are capable of pivoting in a single plane (e.g., one-way and two-way articulation) or two nominally perpendicular planes (e.g., four-way and all-way articulation). An alternate method employs a softer and more flexible shaft material at the distal end of the device without the use of vertebrae. This method of articulation results in deflection of the distal tip of the device similar to that accomplished by articulation, but with less control over the direction or tracking (the ability to move the distal tip within a well-defined plane), and a lower angle of deflection. Articulation angles can be higher than 90 degrees when vertebrae are employed; without vertebrae, however, articulation is generally limited to less than 90 degrees of deflection.

Articulation Wire:

Articulation wires are typically attached to the distal tip of the tool, pass through an articulation section (e.g., vertebrae, spring guides, guide tubes), pass down the length of the shaft (sometimes through lumen in an extrusion, or through spring guides—flexible springs that will bend but not compress when the articulation wires are stressed), and ultimately to the proximal (handle) end where they are attached to a gear system. These wires typically range in diameter from about 0.008″ to 0.027″. These wires are typically made of steel or other metal alloys, but other materials such as Kevlar, Nitinol, nylon, rayon, and other polymer materials, as well as combinations of these materials can be used. The wires need to have minimal stretch to ensure that the articulation can be controlled. Typical elongation percentages for wire range from 1% to 4%.

Drums:

The articulation wires are connected to drums within the proximal end of the tool. These drums can range in diameter from about 0.5″ to 2″ depending on the application. The larger sizes are needed when large articulation angles are desired or long tool working lengths are used (longer lengths of tools require larger drums to take up the stretch in the articulation wire). The shape of the drum may also vary depending on the application. A cam shape may be desired to give the operator a mechanical advantage or to change the rate at which the distal end articulates during use. The drums are typically rotated 30 to 60 degrees in each direction, for a typical rotational range of 60 to 120 degrees. This rotation wraps the articulation wire around the circumference of the drum or cam, pulling on the distal articulated end of the device. This angle depends on the size of the drum and the application of the tool. Alternatively, the articulation wire may be pulled by a rack and pinion system, cam drive, planetary gear system, etc., determined by the force and travel required by the application.

Gear System:

A gear system is typically connected to each articulation drum. This can serve several purposes. First, a 90 degree rotation of one joystick axis may be desired so that both drums are directing the articulation wires along the tool's axis, in such a way as to have all four articulation wires parallel. Second, this gearing can be used to create a mechanical advantage such that less effort is needed when applying manual force to the joystick lever. Third, the gear ratio can be changed to allow a smaller diameter drum to be employed, but this increases the torque required to rotate that drum. A similar reduction can be accomplished using a planetary gear or rack and pinion mechanism.

Joystick Mechanism:

The joystick mechanism consists of a joystick lever which, when the user applies manual pressure, will either directly rotate one of the drums or rotate the arc arm which in turn will drive the gear system, thereby rotating the other drum. A universal swivel joint is located at the end of the joystick lever. This joint allows movement in one direction without effecting the other direction, thus allowing the drums to be rotated independently or simultaneously by the joystick lever, thereby providing all-way articulation rather than just four-way articulation along each plane. The length of the joystick lever can vary depending on the application of the tool. The movement of the joystick lever is limited by physical stops that are set by the assembler to ensure that the articulation will not damage the parts or other devices in contact with the articulating end. The joystick lever is typically moved (translated, displaced) 30 to 60 degrees in any one direction before hitting one of these stops. These stops can consist of limit screws, shaft collars, or other mechanical devices that will limit the joystick's, gears', and/or drums' ability to travel beyond a predetermined position.

FIG. 3 shows the preferred embodiment of the joystick device for the invention. This joystick device is disclosed in parent application Ser. No. 10/462,951, filed on Jun. 17, 2003, incorporated herein by reference. Movement of joystick 110 in the up/down plane causes rotation of shaft 120 and drum 130. Up/Down articulation wires 140 are thereby pulled/pushed a distance proportional to the up/down movement of joystick 110. Movement of joystick 110 in the left/right plane causes rotation of arc arm 150, which translates this movement to shaft 160. Shaft 160 is attached to gear 170, which turns gear 172, which translates the rotation of shaft 160 by 90 degrees. Gear 172 further rotates drum 180, which pushes/pulls the left/right articulation wires 190. Movement of joystick 110 in the up/down plane thus causes tip articulation in only one plane (up/down), while joystick motion in the perpendicular right/left plane causes tip articulation in only the perpendicular right/left tip plane. Joystick motions that are not confined to a single plane cause motions of the tip in both planes. Since the joystick can be moved in two axes translationally, and in 360 degrees rotationally, the tip can be moved anywhere along its sphere. The tip motion is thus fully intuitive. Also, since the tip is moved fully mechanically, there is tactile feedback from the tip to the user's thumb operating the joystick, which helps to detect obstructions and the like.

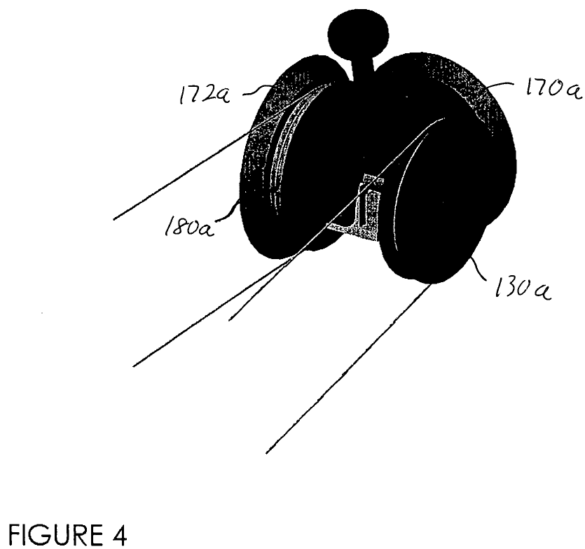

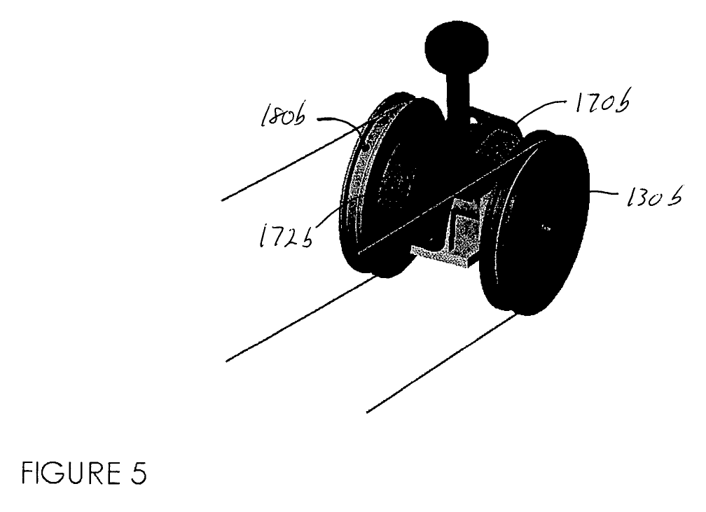

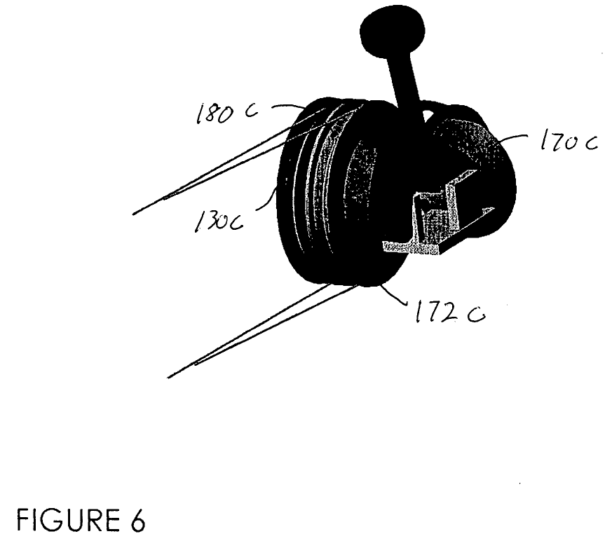

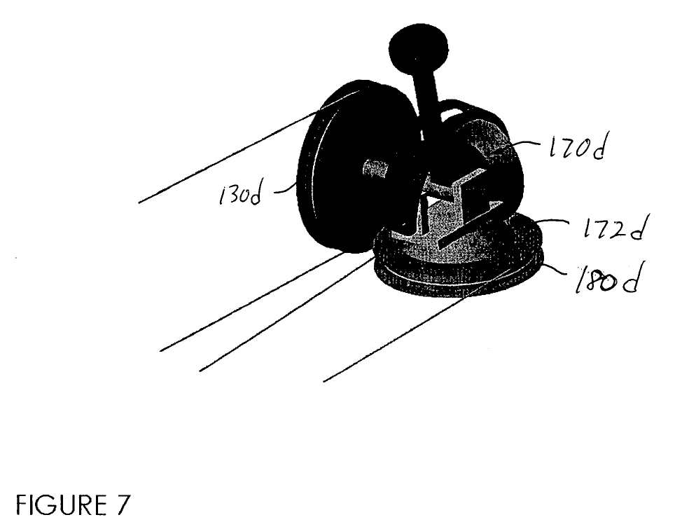

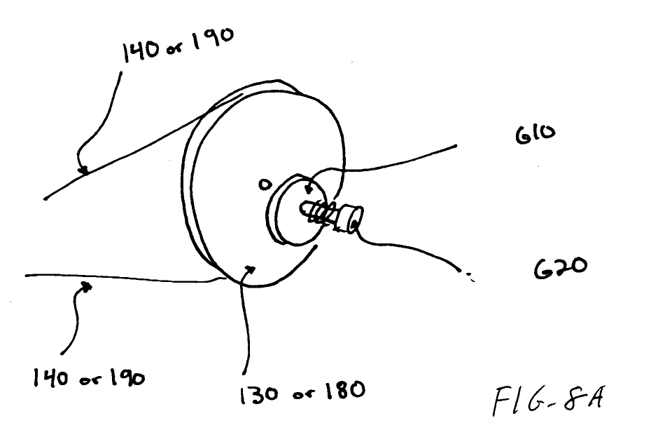

FIGS. 4 through 7 show other possible configurations for the inventive mechanism. FIG. 4 shows directly intermeshed gears 170a and 172a, with drum 180 coupled to gear 172a. FIG. 5 is very similar, but with intermeshed gears 170b and 172a inside of rather than outside of drums 130b and 180b. FIG. 6 shows a configuration in which the drums 130c and 180c are together. FIG. 7 shows a configuration in which drums 130d and 180d are in different planes. In this embodiment, the second gear 172d can be integral with drum 180d.

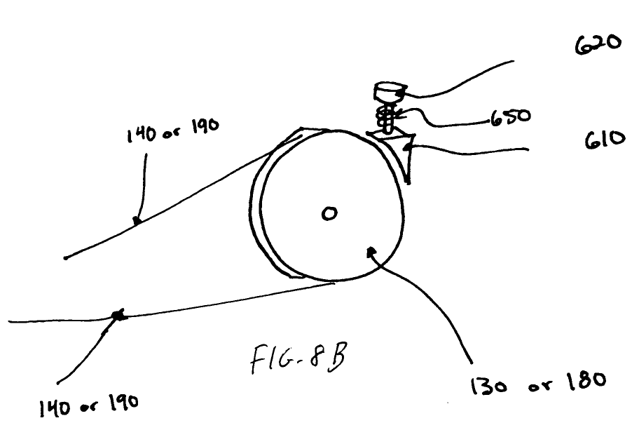

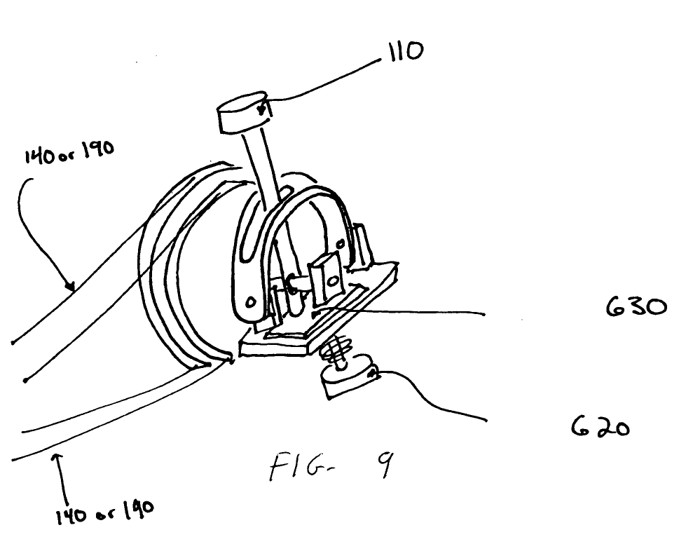

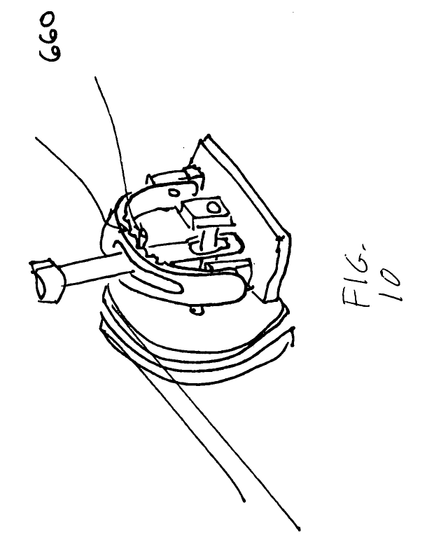

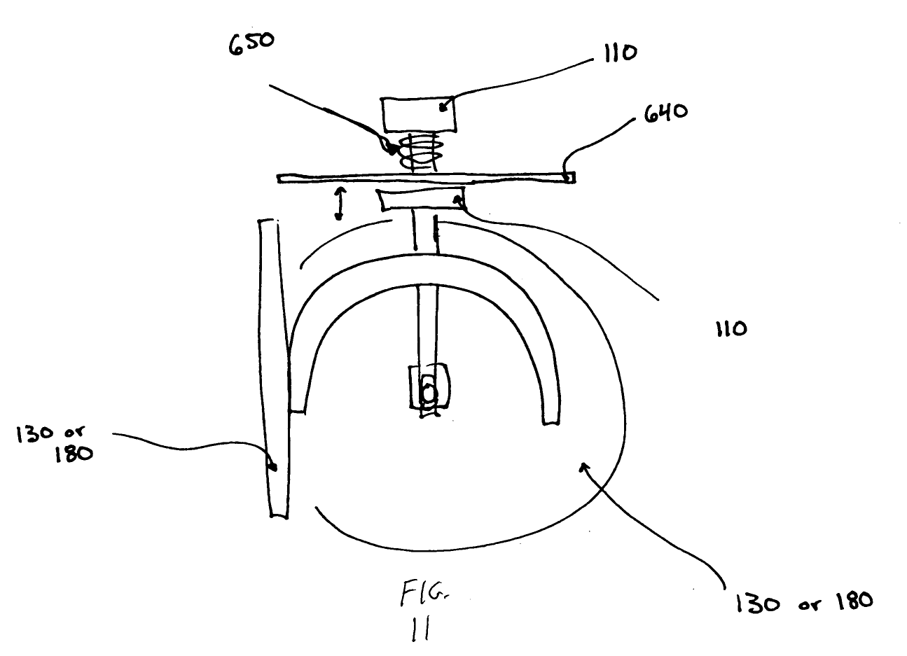

A braking mechanism is also included in the invention in which the articulation means is frozen or held in a particular position. This braking mechanism can take the form of: a friction brake (FIGS. 8A and 8B) in which a pad 610 is forced to contact the joystick 110, one or both of the drums 130 and 180, or one or both of the gears 170, 172; pushing the joystick down (FIG. 9), and latching this position, into a soft material 630 (e.g., a rubber pad) that holds the joystick position until the latch 620 is released; a ratchet mechanism 660, FIG. 10, on the gears and/or drums; or forcing the joystick up into a pad 640, FIG. 11 (e.g., a pad of soft rubber) via a spring 650, in such a way as to stop the joystick's movement until the joystick is pushed down (away from) this pad and allowed to move freely.

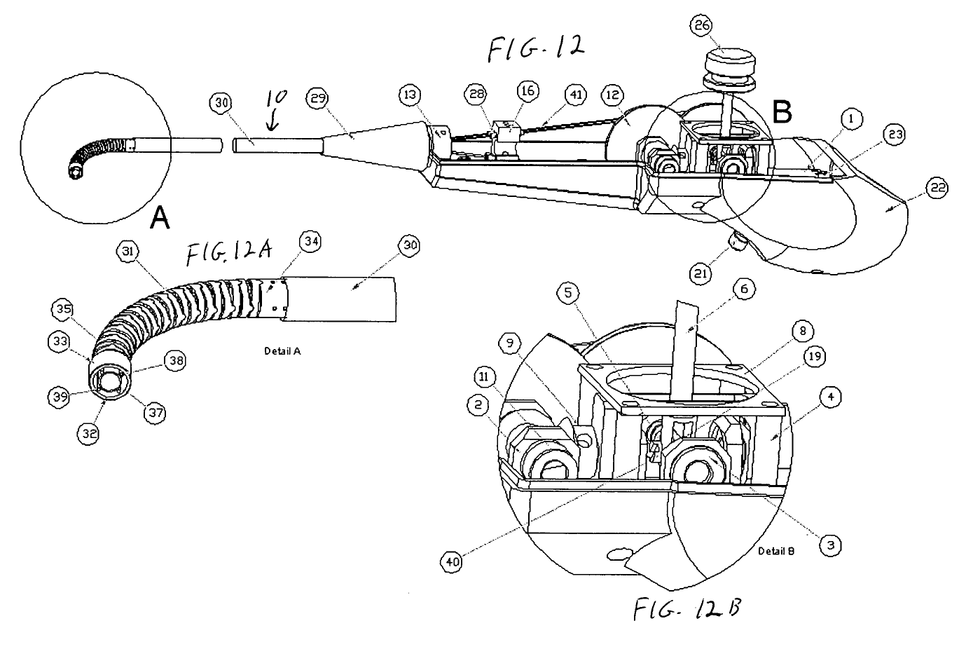

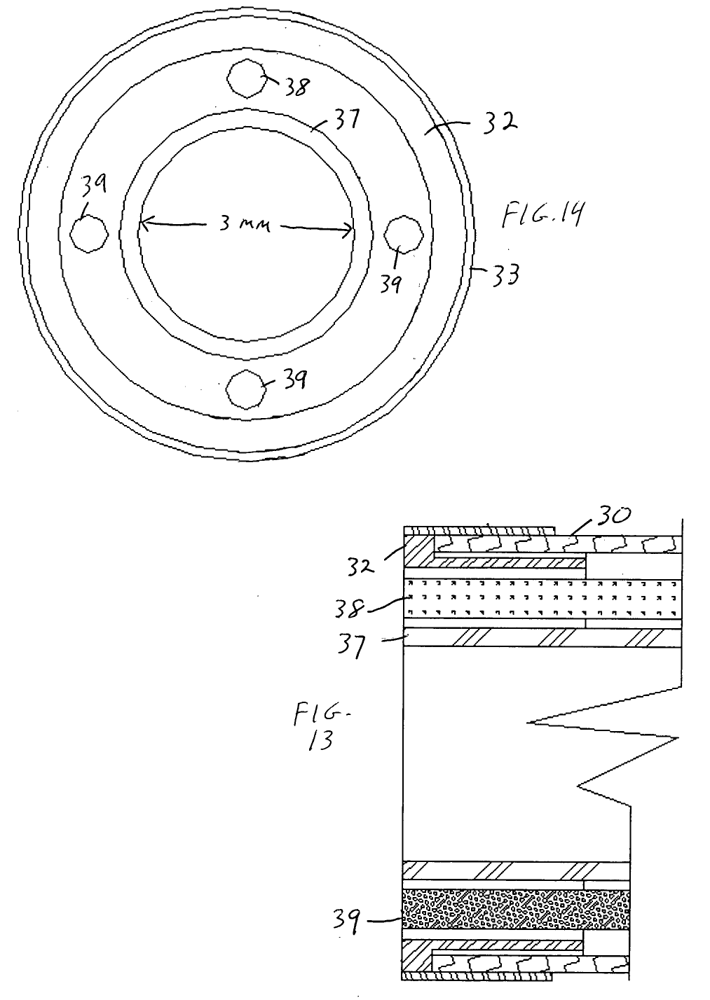

The current embodiment of the invention employs a 6 mm diameter fiberscope used for the visual examination of remote areas. This scope could also be a rigid multi-lens borescope or a video scope employing an imaging sensor (such as a CCD, CID, or CMOS sensor) at its distal tip, in place of a coherent image bundle for transmitting the visual image from the distal tip of the scope to the proximal end of the scope, or some other remote viewing location. In addition to collecting a visual image of the area of interest, the fiberscope has a 3 mm diameter working channel that permits the passage of small NDT probes such as eddy current probes and ultrasonic transducer probes, and the like (for example an electrochemical fatigue sensor probe, or electrical other sensors), remediation tools such as grinding tools or light guides to deliver laser light (that can be used to melt and/or fuse an area around a crack in order to repair the crack and/or relieve stress in the area), as well as a second scope of small enough diameter to fit through the working channel in order to view more remote locations that the larger diameter scope cannot navigate or transverse. The preferred embodiment of this invention is shown in FIGS. 12, 12A and 12B, which depict the main components of the scope employed for delivering these NDT probes and remediation tools (some standard features are not shown in detail).

Description of NDT Scope Assembly Items in FIGS. 12, 12A and 12B

| Item # | Description |
|---|---|
| 1 | BASE PLATE |
| 2 | BEARING BLOCK |
| 3 | BEARING |
| 4 | STANDOFF |
| 5 | PULLEY ARM |
| 6 | ARM COUPLER |
| 8 | STOP PLATE |
| 9 | GEAR MODIFIED |
| 10 | SHAFT LONG |
| 11 | ES0302 SHAFT COLLAR |
| 12 | ES0300 DRUM |
| 13 | SHAFT COUPLER |
| 16 | SPRING GUIDE BLOCK |
| 19 | WASHER TEFLON |
| 21 | ACMI CONNECTOR |
| 22 | HANDLE BOTTOM |
| 23 | SPACER |
| 26 | JOYSTICK CAP |
| 28 | SPRING GUIDE STOP |
| 29 | STRAIN RELIEF |
| 30 | MASTER SHEATHING |
| 31 | VERTEBRAE LINK |
| 32 | HEAD DISTAL |
| 33 | SLEEVE HEAD |
| 34 | SPRING GUIDE COLLAR |
| 35 | VERTEBRAE LINK MOD |
| 37 | WORKING CHANNEL |
| 38 | QUARTZ IMAGE GUIDE |
| 39 | LIGHT GUIDE FIBER |
| 40 | COUPLER SCREW |
| 41 | CONTROL WIRE |

Referring to FIGS. 12, 12A and 12B, handle 22 contains a mechanical joystick articulation mechanism of the type described above. The articulation mechanism uses control wires 41 to cause the bending section 33 at the distal tip to articulate. The shaft 10 mechanical structure is composed of: a stainless steel monocoil (not shown) to provide hoop strength to the member, a stainless steel wire braid (not shown) to provide torsional stability to the assembly and to prevent stretching of the shaft, a sheath 30 of polyurethane covering the braid and monocoil to prevent atmospheric contaminants (dust, water, oil, etc.) from entering the shaft, and an external tungsten braid (not shown) to provide abrasion resistance to the shaft, protecting the soft underlying polyurethane. Within the 6 mm diameter shaft 10 is a 3 mm internal diameter working channel 37 that extends from the distal tip, through the length of the shaft, and terminates at the handle. This working channel is used to guide NDT probes and remediation tools down the scope to the remote area of interest. The working channel is constructed from a 90A durometer urethane material that encapsulates a 0.1 mm thick stainless steel monocoil (not shown), which prevents the working channel material from kinking. Also contained within the shaft 10 are light guides 39 for transmitting the source light from the light source to the object, and quartz image bundle 38 for transmitting the image of the object from the distal tip to the proximal end of the scope where it is imaged onto a CCD camera.

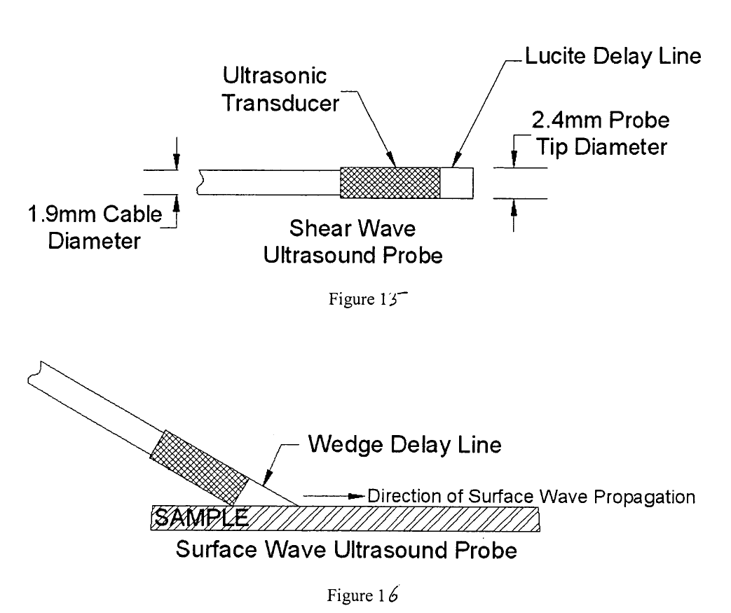

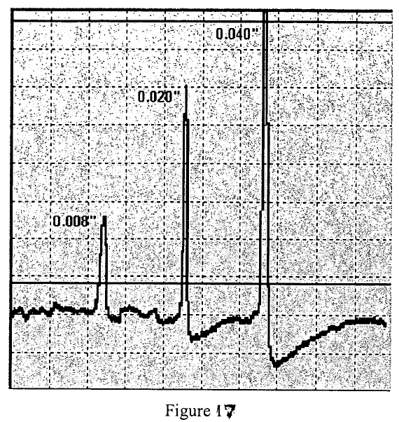

NDT probes, such as ultrasonic transducers and eddy current probes, can be manufactured to pass through the 3 mm working channel, while maintaining an adequate signal-to-noise ratio. The figures depict two versions of ultrasonic probes that were constructed to measure the thickness of a sample (FIG. 15) and the presence of surface cracks (FIG. 16). Both probes have a maximum outer diameter of 2.4 mm, which permits their passage down the 3 mm working channel of the 6 mm scope. In addition, both probes are compatible with commercially available ultrasonic electronics such as the portable, battery operated, Stavely Sonic 1200HR.

The shear wave probe (FIG. 15) has a thickness measurement range of 0.25-4.5 mm, and is made from a 2 mm diameter transducer with a 10 MHz operating frequency. The thickness range of the probe can be adjusted by changing the operating characteristics of the transducer.

The surface wave probe (FIG. 16) projects an ultrasonic pulse along the surface of the sample. Therefore, it is capable of detecting surface breaking cracks and voids that lie in front of the probe tip, dramatically increasing the area of inspection compared to shear wave transducer devices.

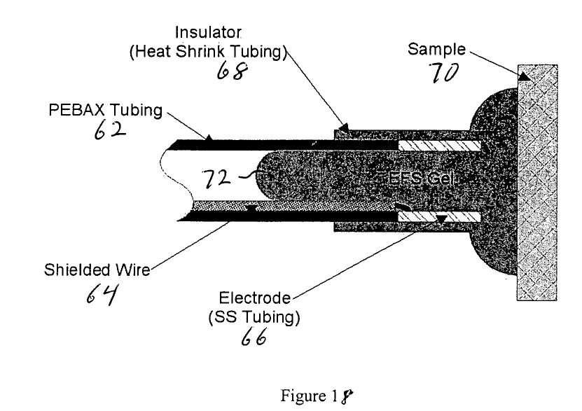

Another means of defect verification employs eddy current probes. A 2.5 mm diameter eddy current probe with a 1 MHz operating frequency was couple to commercial Staveley Nortec 2000S electronics for signal processing. Both relative and absolute probes were constructed, with an absolute probe without radial shielding yielding the best results. This probe was the least sensitive to lift-off error (moving the probe away from the sample surface), and could be used at a wide range of angles between the probe and sample surface. Crack detection of cracks having a depth of less than 0.1 mm is easily accomplished with this probe as can be seen in FIG. 17 that depicts a scan over a crack standard plate having cracks of three depths as indicated in the drawing.

The Electrochemical Fatigue Sensor (EFS) electrode (FIG. 18) is delivered through the 3 mm working channel through a 2 mm OD PEBAX® fluoropolymer tube 62. PEBAX® is available from Zeus Industrial Products, Orangeburg, S.C. Running the length of the tube 62 is a shielded wire 64, which is soldered to a 2 mm OD stainless steel tube 66. The stainless tubing 66 is electroplated with a thin coating of platinum black to improve its conductivity and increase its surface area. Around the stainless tubing 66 is a 6 mm length of heat shrink tubing 68 that prevents the conductive surface of the probe (the stainless steel 66) from coming into direct contact with the sample 70, as this would cause a short in the electrical circuit. Therefore, the only conductive path from the electrode 66 to the sample 70 is through the EFS electrolyte gel 72.

While this embodiment of the invention utilizes a 6 mm OD shaft in order to access small diameter openings, such as a borescope inspection port on an aircraft engine, other diameter scopes with working channels can be envisioned. In these embodiments, where the scope diameter exceeds 6 mm, larger diameter working channels can be accommodated, permitting the use of larger inspection devices and remediation tools.

Other embodiments will occur to those skilled in the art and are within the following claims.

What is claimed is:

1. An endoscope for remotely viewing and testing relatively inaccessible regions of structures that are under stress when used, the endoscope having a mechanically-articulated articulating distal end and comprising:
a. an elongated shaft having a proximal end and a distal working end;
b. a working channel located within the shaft and open at both ends;
c. a remote material stress testing or viewing probe located at least partially within the working channel, and adapted to be exposed to the region to be tested;
d. at least one light guide in the shaft for carrying light introduced into the proximal end to a remote viewing area proximate the distal end; and
e. a user-operable shaft tip steering mechanism for articulating the distal end of the shaft comprising:
at least two rotatable drums;
at least a pair of wires coupled to the drums, and also coupled to the tool's articulating distal end, for translating drum rotation into distal end articulation;
a mechanical joystick moveable translationally and through 360 degrees rotationally; and
a mechanism coupling the joystick to the drums, that mechanically translates motion of the joystick into rotation of the drums, wherein motion of the joystick in one plane causes rotation of only a first drum, and motion of the joystick in a perpendicular plane causes rotation of only a second drum, and movements of the joystick not wholly within these two planes causes rotation of both the first and second drums.

2. The endoscope for remotely viewing and testing relatively inaccessible regions of structures that are under stress when used of claim 1 wherein the mechanism coupling the joystick to the drums comprises:
a rotatable shaft coupled to one drum and coupled to the joystick;
an arc arm rotatable about an axis transverse to the shaft axis by movement of the joystick in a plane transverse to the first plane; and
a gear system for translating rotation of the arc arm to rotation of a second drum.

3. The endoscope for remotely viewing and testing relatively inaccessible regions of structures that are under stress when used of claim 2 wherein the arc arm defines an opening through which the joystick passes.

4. The endoscope for remotely viewing and testing relatively inaccessible regions of structures that are under stress when used of claim 3 wherein the joystick is coupled to the shaft through a universal swivel joint.

5. The endoscope for remotely viewing and testing relatively inaccessible regions of structures that are under stress when used of claim 2 wherein the gear system comprises a first gear coupled to the arc arm and a second gear coupled to the first gear at an angle to the first gear.

6. The endoscope for remotely viewing and testing relatively inaccessible regions of structures that are under stress when used of claim 2 wherein the drums rotate about essentially parallel axes.

7. The endoscope for remotely viewing and testing relatively inaccessible regions of structures that are under stress when used of claim 1 wherein the probe comprises a shear wave ultrasonic material testing probe.

8. The endoscope for remotely viewing and testing relatively inaccessible regions of structures that are under stress when used of claim 1 wherein the probe comprises a surface wave ultrasonic material testing probe.

9. The endoscope for remotely viewing and testing relatively inaccessible regions of structures that are under stress when used of claim 1 wherein the probe comprises an eddy current material testing probe.

10. The endoscope for,remotely viewing and testing relatively inaccessible regions of structures that are under stress when used of claim 1 wherein the probe comprises an electrochemical fatigue sensor material testing probe.

11. The endoscope for remotely viewing and testing relatively inaccessible regions of structures that are under stress when used of claim 1 wherein the probe comprises a second endoscope which fits within the working channel.

12. An endoscope for remotely viewing and accessing relatively inaccessible regions of structures, the endoscope having a mechanically-articulated articulating distal end and comprising:
a. an elongated shaft having a proximal end and a distal working end;
b. a working channel located centrally within the shaft and open at both ends;
c. a plurality of light guides in the shaft and spaced circumferentially around the outside of the working channel, for carrying light introduced into the proximal end to a remote viewing area proximate the distal end;
d. an image guide in the shaft and spaced from the light guides outside of the working channel, for carrying an image to the proximal end of the shaft, for viewing by the user directly or using a camera; and
e. a user-operable shaft tip steering mechanism for articulating the distal end of the shaft comprising:
at least two rotatable drums;
at least a pair of wires coupled to the drums, and also coupled to the tool's articulating distal end, for translating drum rotation into distal end articulation;
a mechanical joystick moveable translationally and through 360 degrees rotationally; and
a mechanism coupling the joystick to the drums, that mechanically translates motion of the joystick into rotation of the drums, wherein motion of the joystick in one plane causes rotation of only a first drum, and motion of the joystick in a perpendicular plane causes rotation of only a second drum; and movements of the joystick not wholly within these two planes causes rotation of both the first and second drums.

13. The endoscope for remotely viewing and accessing relatively inaccessible regions of structures of claim 12 wherein the working channel comprise a urethane-based tube.
